(19) United States
(12) Patent Application Publication
(10) Pub. No.: US 2008/0085307 A1
(43) Pub. Date: Apr. 10, 2008

(54) Paired/matched medication

(76) Inventor: Renjit Sundharadas, San Diego, CA (US)

Correspondence Address: Dr. Renjit Sundharadas, M.D., 11454 Raedene Way, San Diego, CA, 92131, US

(21) Appl. No.: 11/495,126

(22) Filed: Jul. 29, 2006

(51) Int. Cl. A61K 9/20

(52) U.S. Cl. 424/464

(57) ABSTRACT

This patent pairs/matches medication components. This may help with more accurate dosing when the need arises to split tablets. Also there may be increased patient compliance with patients taking many medications. Additionally, this patent would function competition to combination patents, thus driving down the cost for consumers.

### CROSS-REFERENCE TO RELATED APPLICATIONS

This application cross references a previous provisional patent application filed on Jul. 30, 2005. The USPTO number is 60/703790. The number 113264 is also listed on the return postcard.

This patent addresses the need to pair or match two or more subunits of medication by using a vector to hold them together for the following reasons. First, this would allow a combination of medication that could be more tailored to a patient's need. This combination could be novel. Additionally, this patent would cover a situation where there are two or more subunits of medication that contain identical or readily identifiable doses. Thus the patient could remove the vector and get accurate partial doses. The subunits could also be readily identifiable so that they could be taken separately. Novel pairing/matching of medication could also occur as prescribed by a physician. In addition, this patent expands competition with combination medications. By definition, combination medications have components which can't be separated. Since this patent has subcomponents that can be separated, it is unique but competes for the same marketplace. Thus, this would drive down prices of combination medications. Also, this would help non-profitable combinations that are still useful make it to the market. There are other uses too. For example, it would also enable pharmacies to stock only the subunits of the medication and combine them as needed. This is especially useful when narcotics of varying doses are combined with acetaminophen of various doses.

### BACKGROUND

A review of the patent literature does not show any instances where subunit medications are paired or matched so that one or more medications can be taken together without a patient having to worry about the matching processes themselves or if 2 closes of a single medication are matched, the patient wouldn't have to worry about dividing a medication to get accurate dosing. Matching medications would have several advantages. There are pre-existing patents with a goal of making an easy or accurately divisible tablet; these patents have to do with making capsules or tablets that are easy to split, introducing error, or difficulties in production of medication. Error is introduced because there is no guarantee that the tablets or pills will split 100% evenly. Thus with matched pre-divided tablets, one could simply remove the vector holding together the tablets and thus have pre-divided accurate doses. In addition, using a vector to hold together pre-divided medication would give the ability to match a variety of different medications giving the physician, patient or pharmacist the ability to give novel pharmaceutical products that do not exist on the market place. This could be done by machine or by hand. The patient would be ingesting fewer vectors than the comparative number of tablets or capsules, thus increasing patient compliance. Also, pharmaceutical companies are known to introduce combination medications in order to prolong competitive advantages. However, in most cases they are simply combining generic products in a way that gouge the public forcing patients to buy 2 or more generics and then to take them together. A matched, pre-divided tablet would circumvent this abuse since a combination medication patent by definition is not separable into individual components whereas a matched pre-divided tablet is separable into individual components so that one could simply pair two or more generics together to get the equivalent of a proprietary combination medication. This would save elderly, indigent, as well as society in general a good deal of money and eliminate the need for them to buy different generics and take them together as this would be a more simplified process. Also, a pharmacist or patient would be able to easily separate components so that different medication can be separated as needed or the dose can be reduced so that a patient wouldn't have to cut pills, etc. This would have production advantages as the producing company could produce fewer dosages of tablets knowing that they can be accurately divided or paired and thus gaining a competitive advantage. Patients could be issued a device to separate the vector from the pill. This would be a device analogous to a pill cutter.

### BRIEF SUMMARY OF THE INVENTION

As stated above, this invention pairs/matches two or more medication subunits which are held together by a vector. The vector may also function as a medication subunit. Placebo is considered a medication subunit. The medications could be generic or proprietary. The benefits of this invention are listed throughout this patent application. If one were to define a caplet as a tablet shaped like a capsule, then an example would be to combine two half-caplets of medication and encase it in a capsule which would function as a vector.

### DETAILED DESCRIPTION OF THE INVENTION

The following description is one interpretation of the patent claims. Assuming that a caplet is a tablet in the shape of a capsule, if it was halved down the middle, one would have 2 half-caplets. This invention would be in this case a pairing of two medication subunits in the shape of half-caplets that would be encased in a capsule. The capsule is thus the vector and the end creation would be similar in appearance to a normal capsule.

In the case of a capsule, in one possibility, it could be filled with components chat appear to be either quarter caplets so that up to four different medication subunits can be matched.

The medication subunit may be generic or proprietary. Because there are so many possible medications on the market it is assumed that there are many aspect of these medications that would be obvious when used in this patent. Such obvious aspects include:
1. Any medication component or the vector itself may function or have an enteric coating.
2. Any medication components may be chewable.
3. Vector or components or both can be designed to survive acidic environments such as the stomach and release medication after that environment has passed.
4. Vector or components or both can liberate one or more medications at different rates.
5. Specific components may be designed to release medication or other substances only if they are crushed, thus going through the digestive system unreleased if not crushed.
6. Medication components may be divisible.
7. The packaging may contain specific information about what matched medications have been issued and any relevant product information about the vector, components or effects of matching the components.
8. Components may be medications that have been used in the past, present or yet to be used in the future.
9. Any medication component of the vector itself may be immediate release. short/long acting, extended release, delayed release, quickly dissolving or have any other pertinent attribute of medications on the market.
10. A flavoring, coloring or any other secondary use may be added as a component or may be present in the vector itself.
11. Flavoring or flavoring components may be added for patient satisfaction, especially with regards to compliance in children. This can be added to the components, vectors or both.
12. Each component can have individual labeling on the component with regards to any information related to the medication including, but not limited to dose, medication name, maximum dose, side effects, contraindications, and other information. This labeling may also be on the vector itself or both the component and the vector.
   In Practice, examples may be as follows:

### EXAMPLE

1. A pharmacist or patient has an 80 mg paired medication with 2 halves composed of identical medication. To obtain a 40 mg dose, the vector simply has to be removed and then one has two 40 mg doses per tablet. The vector could be easily removed by using a device that grabs each end of the vector, pulls it apart and then the vector is ejected from the device.

2. There is a need to match two medications, say anti-hypertensives, and there is no combination medication. The two components could be matched using this technology.

3. Medications are taken by a patient once per day. These medications can be matched into one vector, thus increasing compliance and making it simpler for the patient to remember to take one medication than to go over many bottles.

4. An example of a matched medication is two subcomponents that are produced as pills that essentially are half of pills. Thus these two would be combined in a capsule that could be easily removed.

5. Examples of a matched medication of more than 2 subcomponents are pills produced as discs, or small tablets that essentially would fit in a capsule as a vector. Thus these components would be combined in a capsule that could be easily removed.

1. Any pairing/matching of one or more medication subunits that are held together by a vector (which may also function as a medication subunit) so that the patient can take the medication as one unit.

2. Dosing of any subcomponent can be in the range of 0.00000001-1000000000 mg per tablet.

3. Subunits may be readily identifiable by taste, size, color, weight, markings, writing, texture, or shape.
